SEQUENCE LISTING (24 sequences)

SEQ ID NO: 1 - protein, 334 aa, Yarrowia lipolytica
MKLSTILFTACATLAAALPSPITPSEAAVLQKRVYTSTETSHIDQESYNFFEKYARLANIGYCVGPGTKIFKPFNCGLQCAHFPNVELIEEFHDPRLIFDVSGYLAVDHASKQIYLVIRGTHSLEDVITDIRIMQAPLTNFDLAANISSTATCDDCLVHNGFIQSYNNTYNQIGPKLDSVIEQYPDYQIAVTGHSLGGAAALLFGINLKVNGHDPLVVTLGQPIVGNAGFANWVDKLFFGQENPDVSKVSKDRKLYRITHRGDIVPQVPFWDGYQHCSGEVFIDWPLIHPPLSNVVMCQGQSNKQCSAGNTLLQQVNVIGNHLQYFVTEGVCGI

SEQ ID NO: 2 - DNA, 1005 bp, Yarrowia lipolytica
atgaagctttccaccatcctcttcacagcctgcgctaccctggctgccgccctcccttcccccatcactccttctgaggccgcagttctccagaagcgagtgtacacctctaccgagacctctcacattgaccaggagtcctacaacttctttgagaagtacgcccgactcgcaaacattggatattgtgttggtcccggcactaagatcttcaagcccttcaactgtggcctgcaatgtgcccacttccccaacgttgagctcatcgaggagttccacgacccccgtctcatctttgatgtttctggttacctcgctgttgatcatgcctccaagcagatctaccttgttattcgaggaacccactctctggaggacgtcataaccgacatccgaatcatgcaggctcctctgacgaactttgatcttgctgctaacatctcttctactgctacttgtgatgactgtcttgtccacaatggcttcatccagtcctacaacaacacctacaatcagatcggccccaagctcgactctgtgattgagcagtatcccgactaccagattgctgtcaccggtcactctctcggaggagctgcagcccttctgttcggaatcaacctcaaggttaacggccacgatcccctcgttgttactcttggtcagcccattgtcggtaacgctggctttgctaactgggtcgataaactcttctttggccaggagaaccccgatgtctccaaggtgtccaaagaccgaaagctctaccgaatcacccaccgaggagatatcgtccctcaagtgcccttctgggacggttaccagcactgctctggtgaggtctttattgactggcccctgatccaccctcctctctccaacgttgtcatgtgccagggccagagcaataaacagtgctctgccggtaacactctgctccagcaggtcaatgtgattggaaaccatctgcagtacttcgtcaccgagggtgtctgtggtatctaa

SEQ ID NO: 3 - protein, 498 aa, Yarrowia lipolytica
MPLELPSLNASIVGNTVQNGAVEQFLNIRYADIPGKFEKPVLKNDWNGAEIDATKVGPVCPQPRTPFNFFSVPDDLWEKVNVDTYQDGLLCDNLIVTRPKGVSANARLPTVVWIHGGSNIEGSIYNLIYEPQFLVAESVRVGKPIVHVCIEYRLGLAGFLTKNGKGNWGTWDQYTGCQWVNRHIQDFGGDPLNVTLTGESAGSVAVHNMLIKDSMNGRKLFRNAVMMSGTLETITPQPPKWHARLEEKVAKVTGKEVADLASLSDKELLDAQIKLNVAVCMTCDDGDFFEPGWKQHLTPDWLDKLIISDCKDEGMLYFLPVNAQDDEELLAKVAKSPVGKEISELYGIKEGGDIKSACLDLKTDATFNYFNHLLFKKMEEARNNGSTSRVYRLAVDEPNPHNPDQRAHHAVDVLYMFNSTKFNEHGDKLSRLFQSHFLRLAYGLEPWDHRNFGVYRNGGYQQLPLSELNKVRPVERYEALSKMDFGQVGRLSNALSRL

SEQ ID NO: 4 - DNA, 1497 bp, Yarrowia lipolytica
atgcctctcgaactcccctcgctcaacgcctcgattgtcggcaacaccgttcagaacggcgctgttgagcagtttctcaacatccgatacgccgacattcctggcaagtttgagaagcccgtgctcaagaacgattggaacggcgcggagatcgacgccaccaaggtcggtcccgtgtgcccccaaccccgcaccccattcaacttcttctccgtgccagacgacctctgggagaaagtcaatgtggacacgtaccaggacggtctgctgtgcgacaacctgattgtgacgcgaccgaagggcgtgtctgccaacgcccggctgcccactgttgtgtggatccacggcggctccaatattgagggcagtatctacaacctcatctatgagccccagttcctggtggcagagtcggtgcgagtaggcaagccgattgtgcacgtgtgtatcgagtaccgactgggtctcgcgggcttcctcaccaagaacggcaagggcaactggggcacgtgggatcagtacacgggctgccagtgggtcaaccgccacattcaggactttggaggcgatcctttgaacgtgacattgaccggtgagtctgccggctctgtagcagtccataacatgctcatcaaggactccatgaacggccgaaagttgttccgaaatgccgtcatgatgagtggcactctcgagaccatcactcctcagcctcccaagtggcatgctcgtttggaggagaaggtggccaaggtcactggcaaggaagtggccgaccttgcttctctgtccgataaggagctgctcgacgcccagatcaagctcaatgtggctgtgtgcatgacttgcgacgacggcgactttttcgagcccggatggaagcagcatctgactcctgactggctcgacaagctcatcatctccgattgcaaggacgagggcatgctgtatttcctgccagtcaacgcgcaggacgacgaggagctgttggcaaaggtggccaagtcgcccgtgggtaaggagatttccgagctttacggcatcaaggagggtggcgatatcaagtctgcgtgtctcgatctcaagactgacgccaccttcaattactttaaccatctgctgttcaagaagatggaggaggcccgaaacaacggctccacttctcgagtttaccgtctggccgtcgatgagcccaacccccacaaccccgaccagcgggcccaccacgccgtcgacgtgctgtacatgttcaactcgaccaagttcaacgagcacggcgacaagctgtctcggctgttccagagccactttttgcggctggcgtatggcctggagccctgggaccatcgaaactttggagtgtacagaaacggcggctaccagcagctgccgctgagtgagttgaacaaggtccgacccgtcgagcggtacgaggcgctgtccaagatggactttggccaggttgggcgtttgtccaatgcgctttcgcgcctatga

SEQ ID NO: 5 - protein, 371 aa, Yarrowia lipolytica
MVSLSARIKDFFSVLLLGAATITPSTQTAGVSQGFYDFARDFAHLSNIAYCVNAPITPLNPDFTCGNSCKHFPEIELVKTFGGNFFKTSITGYLAVDHVKKEKYVVFRGTFSLADAITDMQFQLSPFLVDVPALNTFSANDTTAEAQTHCEGCKIHDGFSKAFTETWGNIGEDLQKHLDANPDYQLYVTGHSLGAAMALLGATSIKLKGYDPILINYGQPRVGNKPFAEFINKLWFGEGNGLEITPERKLYRMTHWNDIFVGLPNWEGYTHSNGEVYINNRFINPPLKDVISCAGGENSKCYRSSFSLLSQINLLQNHLAYIDYIGYCALNIGRRELADQEHYTGPYYYGHRSEEDFKKLGLELSTPQVEN

SEQ ID NO: 6 - DNA, 1116 bp, Yarrowia lipolytica
atggtatccctctctgctcgaatcaaagactttttttcggtcctcctcctcggagctgcaaccatcactccctccacacagaccgcaggcgtgtctcaagggttctatgattttgctcgggactttgcccatctgtccaacattgcctactgtgtcaatgctcccatcactccactgaacccggacttcacctgtggcaactcgtgcaagcactttccggaaattgagcttgtgaagacatttggaggcaacttcttcaagacctccattacgggctacctggctgtcgatcatgtcaagaaggagaagtacgttgtcttccgaggaaccttttcgctggcagacgcgatcacggacatgcagttccagctgtctcctttcctggtcgatgtgcctgccctgaacactttctcagctaatgacaccaccgcagaggcccagacgcactgtgagggctgcaaaattcacgacggcttctccaaggcctttaccgagacctggggtaacattggtgaggatctgcagaaacacctggacgctaacccggactaccagctgtacgtgactggccattctctgggagctgctatggcccttcttggagctacttccatcaagctcaagggctacgatcccattctcatcaactacggacagccccgagtcggaaacaagcccttcgctgagttcattaacaagttgtggtttggagaaggcaacggtctggaaatcacccccgagagaaagctgtaccgaatgacccactggaacgacatctttgttggcctgcccaactgggagggatacacccactctaacggtgaagtatacatcaacaaccggttcatcaaccctcctctcaaggatgtcatctcttgtgctggaggcgaaaactcgaagtgctaccgatcctcgttcagcctgctgtcccagatcaatctgctccaaaaccacctggcttacattgattacattggatactgcgctctgaacattggtcgacgagagcttgccgatcaggaacattacactggtccttattactatggtcatcgatctgaggaggactttaagaagttgggcttggagctatccaccccacaagttgagaactga

SEQ ID NO: 7 - protein, 406 aa, Yarrowia lipolytica
MAGFNFTFGQVISYLASMLYGQVDATSSSTRIQATQDLYDFTAKFSRLSNIAYCINAPFTPLRTDFTCGESCRYFPDLQLDSVFGGNFSSASTTGYIAYDHKKKEKYIVFRGTFSIPDIITDIQFQTAPWLTSLPTHLIPTKEDFEHKQAILKHYAAENKGLSNLEERQDVVHEDPSLVPKKMDKCENCQIHDGFAKGFNETIEHAGPQIEKFLGNNTDYKMFVVGHSLGAAQAQLFATQFKLLGFDPYMINFGQPRLGNPEFAAYINQLWFNDTGLVVNDARRFYRVTHWNDIVVGVPDWLNYTHSIGEVFIDEESVYPKLDKVVVCEGGENPLCHRGTFNLWSRINFLQNHLAYIFYIGLCAFNIGRRDVLNMPQYQGNFSYQHNIDPNYNYDTKVPTRISKSN

SEQ ID NO: 8 - DNA, 1221 bp, Yarrowia lipolytica
atggcagggttcaacttcactttcgggcaggtgatttcgtacctggcttccatgctctatggacaggtggatgccacttcatcctccactcgaatccaggccacccaggatctgtacgacttcacagccaagttttcgcgactctcaaacatcgcgtactgcatcaatgcccccttcacgcctctcagaacggacttcacctgcggagaaagctgtcggtacttccccgacctccagctggactcagtgtttggtggtaacttctcctcagcctccactaccggctacattgcatacgaccacaagaagaaggaaaagtacattgtgtttcgaggaactttcagtatccctgatatcatcacagacattcaatttcagactgccccttggttgacctctctgcccacgcatctgatccctaccaaggaggactttgaacacaagcaggccatcctgaagcactacgctgccgaaaacaagggtctcagcaacctggaagagcgacaggatgttgtgcatgaagaccctagcctggttcccaagaaaatggacaagtgcgagaactgccagatccatgacggattcgccaagggcttcaacgagactatcgagcatgccggaccccagattgaaaagttcctgggcaataacaccgactacaagatgtttgttgttggccactctctaggagctgctcaggcccagctgtttgctacacagttcaaactgctgggatttgacccttacatgatcaactttggacagcctcgacttggaaaccctgagttcgccgcctacatcaaccagctgtggttcaacgatactggtctggttgtcaatgatgcccgacgattctaccgagtgactcactggaacgatatcgtcgtgggagtgcccgactggctcaattacacccactctatcggagaggtgttcatagacgaggagagcgtttaccccaagctggacaaggtggtggtgtgcgagggaggagagaaccccctgtgccaccgaggaactttcaacctgtggtcacgaatcaacttcctgcagaaccatttggcttatatcttctacattggtctgtgtgctttcaacattggccgaagagacgtgctcaacatgccacaataccagggcaacttctcgtaccagcacaacatcgaccccaactacaattacgataccaaggttcccacccggatcagtaaatcaaactaa

SEQ ID NO: 9 - protein, 535 aa, Candida rugosa
APTATLANGDTITGLNAIINEAFLGIPFAEPPVGNLRFKDPVPYSGSLDGQKFTSYGPSCMQQNPEGTYEENLPKAALDLVMQSKVFEAVSPSSEDCLTINVVRPPGTKAGANLPVMLWIFGGGFEVGGTSTFPPAQMITKSIAMGKPIIHVSVNYPVVSSWGFLAGDEIKAEGSANAGLKDQRLGMQWVADNIAAFGGDPTKVTIFGESAGSMSVMCHILWNDGDNTYKGKPLFRAGIMQSGAMVPSDAVDGIYGNEIFDLLASNAGCGSASDKLACLRGVSSDTLEDATNNTPGFLAYSSLRLSYLPRPDGVNITDDMYALVREGKYANIPVIIGDQNDEGTFFGTSSLNVTTDAQAREYFKQSFVHASDAEIDTLMTAYPGDITQGSPFDTGILNALTPQFKRISAVLGDLGFTLARRYFLNHYTGGTKYSFLSKQLSGLPVLGTFHSNDIVFQDYLLGSGSLIYNNAFIAFATDLDPNTAGLLVKWPEYTSSSQSGNNLMMINALGLYTGKDNFRTAGYDALFSNPPSFFV

SEQ ID NO: 10 - protein, 534 aa, Candida rugosa
APTATLANGDTITGLNAIVNEKFLGIPFAEPPVGTLRFKPPVPYSASLNGQQFTSYGPSCMQMNPMGSFEDTLPKNARHLVLQSKIFQVVLPNDEDCLTINVIRPPGTRASAGLPVMLWIFGGGFELGGSSLFPGDQMVAKSVLMGKPVIHVSMNYRVASWGFLAGPDIQNEGSGNAGLHDQRLAMQWVADNIAGFGGDPSKVTIYGESAGSMSTFVHLVWNDGDNTYNGKPLFRAAIMQSGCMVPSDPVDGTYGTEIYNQVVASAGCGSASDKLACLRGLSQDTLYQATSDTPGVLAYPSLRLSYLPRPDGTFITDDMYALVRDGKYAHVPVIIGDQNDEGTLFGLSSLNVTTDAQARAYFKQSFIHASDAEIDTLMAAYTSDITQGSPFDTGIFNAITPQFKRISALLGDLAFTLARRYFLNYYQGGTKYSFLSKQLSGLPVLGTFHGNDIIWQDYLVGSGSVIYNNAFIAFANDLDPNKAGLWTNWPTYTSSSQSGNNLMQINGLGLYTGKDNFRPDAYSALFSNPPSFFV

SEQ ID NO: 11 - protein, 534 aa, Candida rugosa
APTAKLANGDTITGLNAIINEAFLGIPFAEPPVGNLRFKDPVPYSGSLNGQKFTSYGPSCMQQNPEGTFEENLGKTALDLVMQSKVFQAVLPQSEDCLTINVVRPPGTKAGANLPVMLWIFGGGFEIGSPTIFPPAQMVTKSVLMGKPIIHVAVNYRVASWGFLAGDDIKAEGSGNAGLKDQRLGMQWVADNIAGFGGDPSKVTIFGESAGSMSVLCHLIWNDGDNTYKGKPLFRAGIMQSGAMVPSDPVDGTYGNEIYDLFVSSAGCGSASDKLACLRSASSDTLLDATNNTPGFLAYSSLRLSYLPRPDGKNITDDMYKLVRDGKYASVPVIIGDQNDEGTIFGLSSLNVTTNAQARAYFKQSFIHASDAEIDTLMAAYPQDITQGSPFDTGIFNAITPQFKRISAVLGDLAFIHARRYFLNHFQGGTKYSFLSKQLSGLPIMGTFHANDIVWQDYLLGSGSVIYNNAFIAFATDLDPNTAGLLVNWPKYTSSSQSGNNLMMINALGLYTGKDNFRTAGYDALMTNPSSFFV

SEQ ID NO: 12 - skipped

SEQ ID NO: 13 - protein, 534 aa, Candida rugosa
APTATLANGDTITGLNAIINEAFLGIPFAEPPVGNLRFKDPVPYRGSLNGQSFTAYGPSCMQQNPEGTYEENLPKVALDLVMQSKVFQAVLPNSEDCLTINVVRPPGTKAGANLPVMLWIFGGGFEIGSPTIFPPAQMVSKSVLMGKPIIHVAVNYRLASFGFLAGPDIKAEGSSNAGLKDQRLGMQWVADNIAGFGGDPSKVTIFGESAGSMSVLCHLLWNGGDNTYKGKPLFRAGIMQSGAMVPSDPVDGTYGTQIYDTLVASTGCSSASNKLACLRGLSTQALLDATNDTPGFLSYTSLRLSYLPRPDGANITDDMYKLVRDGKYASVPVIIGDQNDEGFLFGLSSLNTTTEADAEAYLRKSFIHATDADITALKAAYPSDVTQGSPFDTGILNALTPQLKRINAVLGDLTFTLSRRYFLNHYTGGPKYSFLSKQLSGLPILGTFHANDIVWQHFLLGSGSVIYNNAFIAFATDLDPNTAGLSVQWPKSTSSSQAGDNLMQISALGLYTGKDNFRTAGYNALFADPSHFFV

SEQ ID NO: 14 - DNA, 11606 bp, Artificial Sequence
  plasmid containing Cas9
cgcgtggatcgccggtgcgttgacgttggtgacctccagccagaggtgcccggcgccccgctcgccggcgaactccgtcgcgagccccatcaacgcgcgcccgaccccgtgcccccggtgctccggggcgacctcgatgtcctcgacggtcagccggcggttccacgccgagtacgagatgaccacgaagcccgccaggtcgccgtcgtccccgtacgcgacgaacgtccgggagtccgggtcgccgtcctccccgtcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctagaggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgttaggggcagggcatgctcatgtagagcgcctgccgctcgccgtccgaggcggtgccgtcgtacagggcggtgtccaggccgcagagggtgaaccccatccgccggta

SEQ ID NO: 15 - DNA, 14006 bp, Artificial Sequence
  plasmid containing expression systems for DrBCO, LmATF-S480Q_G409A_V407I_H69A_I484L, and FfRDH
gcggagcctaggccggccagcgtggcgcgtggccatattggccagcagcttactacagctcagccaaagtggatcatgatggacggaaatatcggactggaggccaaaaaggaggtgctcaaatacgcacgggacaagtctgcacaggtggcattcgaacccacgtctgtccccaaagccgctgctctttccgagctcaatctgcccgtttaccccaacaactccattgccttagccacccccaacactgcagagctcgaggccatgttcgaggcattccacgagaagggccgatttgacgttgacgactggtttccagtcatagacggtcttgctctcggagctgactttagaaacggagccaccatgttgtcgcaccaacatcgtggactcaaagctattctcgaacaaggaactctggcccaggcaatccacatgctaccatacatcccaactttgatcatcaagggcggagccaacggcgtcgtcgtcttccaactcattgatgatatcgagtctgccattcattcacagcgttctgcttccaataaaacgcctggcctgttccagaagggcaacgctgcgagtggaaacaccaaggtcggcgtctacatgcagtactttgagcccgaagaagtgggcagtcagtcgattgtaagcgtgactggagctggagacactctgtttggaaccctggccatggagattgtcaaggacgagtcctggttgaacgatatgggagacaagaagagtgcagttgtttctcgagccatgaacaatgctgtgaagactattcagagtaaggacgctgtctgcaagagcatcctttaagtgatttgccatgctttctcttcttccacgatgtaaatacttattttacacactactgtgcagtagcaaatacagaacaagagttgtcgcctattgacagtacagtacgagtagtgtatgtacagtagttatacaatatctatgtgaaattcgtcggcagctttcggctgatgaactacgagttcttcgtaataaatcatcaacgtaataagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgggtgatctcgatgcccgaggaacctctgctcaccccgagctctctgagcgaccttctattatgccttctatgtctgatattcaggacccttctggtgatgacaaggctactccccgaggatctgctgctggtctgccccagcttgagcttgctggacacgcccgacgacttggccaccttgagaacttctttgctgtccaggctcgacagcagatttactcttcttttgctgttttttgtgagtttgacactgcttgttctctcgctcagcttgcttctgctgtgcgaaacgtttgtctttctaacccccttctccttcacactgttgagcctaagcaccctgacatcgctggattctaccactctgacgagtacctttcccgaccttggcctcagcacgattacatgcgagttcttcgagaggttcacgtcgctgacgttgttatgaacggacagaaggagcacgctcacgttgttcgagatgctgttgacgtttttcaggctcacggaaaccaggttacttctgagctccttgagcttatgactcagattgagattcctcacgcttctcagactcgaccctcttggcgacttctctgttttccccacggagaggctaaccgatggcgaacctttgcttttgtttctaaccactgttcttctgatggtctttctggtcttaacttctttcgagatctccagaaggagcttgctcacggccccacctctggtgctcctggtgcccccggagcttccggagttattttcgattacgctcaggacgctgctaccctgcccaagctgccccctcccattgatcagaagctcgattaccgaccttctaagaaggctcttctcggccttctcgctggcaagttcgttcgagagaagctcggttacgtttctgctgctcctcccactacccctacctctgaccttgctcaccctgagggtcaccagtactactgttaccttgttaacgttcccacttcttctgttgcccacattaagactcaggtgcgagagaacgttcctcacaagtgtactctcactccctttctccaggcttgttggcttgtttctctgttcaagtacggtcgagttttttctggttcttggcttgagcgatacaccgatgttcttgttgctatgaacactcgacagcttctccccgaggaccttgagcttcagcgacagtaccgatacggttctaacattggagctgttcgatacaactaccctattgctccccttgacgttcgagataacgatcagaagttctggtcccttgttgagtcttaccgacttgccctttctgatgcccgagataagaacgattacctttacgctcttggtgctcttatgctccctgagatttacgagaagaagaacgttgatgctgttgttaacgataccattcttaaccagcgacgacagggaacccttctttctaacgttggttacgttcgagatgagcagcccactgcttttgctattaagaaccacgttttttctcagggagttggagctaaccgaaacgcttttgttcttaacatttgtgctaccgatcagggtggtcttaacatcgctatttctattgctaagggaacccttgcttctcgacaggagggacaggagctttgtgatatttttaagtctactctccttcgattttaaacgcgtctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatctgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcgtagaccccggtgtatataaaggggtggagaggacggattattagcaccaacacacacacttatactacatgctagccacaaaaatgctctctttcttctggcgaaacggtatcgagactcccgagcccctcaaggctgacgtttccggctctatccctccctggcttcagggaacccttctccgaaacggtcctggtctgttctccgttggcaacacttcctacaagcactggttcgatggtatggctctcattcactccttcacctttaaggatggtgaggttttttaccgatctaagtacctgaagtctgagacttacaagaagaacatcgctgccgaccgaatcgttgtgtctgagttcggaaccatggtgtaccccgatccctgcaagaacattttctcccgagccttctcttacatgatgaacgccattcctgactttaccgataacaacctcattaacatcattaagtacggtgaggattactacgcctcctctgaggtcaactacatcaaccagattgaccccctgacccttgagactctcggacgaactaactaccgaaaccacattgccatcaaccttgccactgctcaccctcactacgacgaggagggtaacacttacaacatgggcactgctattatgaacctcggtcgacccaagtacgtgattttcaaggtgcccgccaacacctctgataaggagaacaagaagcctgccctctctgaggtggagcaggtttgctccattcccatccgaccctccctttacccttcttacttccactcttttggcatgactgagaactacatcatcttcgttgagcaggccttcaagctggacatcgtcaagctggctactgcttacttccgagatattaactggggatcttgccttaagttcgaccaggatgacattaacgtgttccacctggtcaacaagaagactggtaaggctgtgtccgtgaagtactacactgacccctttgttaccttccaccacatcaacgcttacgaggacgatggccacgtcgtcttcgatctcattacttacaaggactctaagctgtacgatatgttctacattcagaacatgaagcaggacgtcaagcgatttattgagactaacaaggacttcgctcagcccgtgtgccagcgatttgtccttcccgtcaacgttgataaggagactcctcaggacatcaaccttgtcaagctgcaggacaccactgccactgctgtcctgaaggaggacggctctgtctactgcacccctgacatcatttttaagggtcttgagctccctgctatcaactacaagtttaactctaagaagaaccgatacttctacggcacccgagtggagtggtccccttaccctaacaaggtcgctaaggtggacgttgttactcgaacccacaagatttggactgaggaggagtgttacccttctgagcctgtctttattgcctcccctgacgccgttgatgaggatgacggtgtgattctttcttctgtggtttctttcaacccccagcgaccccctttcctggttgtcctcgatgctaagtccttcaaggagattgctcgagctaccatcgatgcctctattcacatggaccttcacggccttttcatccacgacaagtctacctaagttttttgatcaatgatccaatggctttcacatacccccccacgcctataattaaaacacagagaaatataatctaacttaataaatattacggagaatctttcgagtgttcagcagaaatatagccattgtaacaaaagccggctatcgaccgctttatcgaagaatatttcccgccccccagtggccaaacgatatcggtcagaaggggcagctctaaacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacatgctagccacaaaaatgaccactaagtacacttccgttcacgagtctcccaacggccctggtgacgctcgacccaccgcttcccagattatcgacgattacaaccttgagggagagctttctggcaagactgttctcgtcaccggctgttcctctggtattggtgttgagactgcccgagctatttaccgaactggtgccaccctttacctcactgcccgagatgtcgataaggccaagaccgttcttcccgaccttgttgacacttcccgagtccactttctccaccttgaccttaactctctggagtctgttcgaggttttgctgagaacttcaagtctaagtccactcagcttcacattctcatcgagaacgctggcgtgatggcctgtcccgagggccgaaccgtcgatggttttgagactcagtttggtatcaaccaccttgctcactttctcctcttttacctcctcaaggatacccttctcaactcttctacccccgctttcaactcccgagttgtcatcctctcttcttgtgctcaccaggctggttccgttcaccttaacaacctgtctcttgagggtggatacgagccttggaagtcttacggccagtccaagactgccaacctttggactgcccgagagatcgagaagcgatttggtgcttccggtatccactcttgggctgttcaccccggttccatcgctactgagcttcagcgacacgtttccgacgagcttaagcagaagtgggctgacgataaggagggtgccaagctgtggaagtccaccgagcagggtgccgccaccactgtccttgctgctgtttcccctgagcttgagggtaagggcggtctttaccttgaggatacccaggttgccaagccccctgcccgaggaatgtttggtgttgctgactgggcttacgatgaggatggcccctctaagctctgggccaagtctcttgagctccttaagctccagtaaaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcacactattatcacattactacatccaaacccctacagggggaggagctctccaatcaaatatgtacattaactatctctcgtaaatcattgttataagaccgtccgtgactgtctaaatcggttcattcgttgtaacaaatcagtgtaacaactcgtggtacgcccggtttgcttcgctcacgctcccctagattttccgtctaggacacaacaagccctggtggatgacgttcaacgccttcagcgcgtctttttcctcaataacacacgagatgttaatttcgttggctccctgagaaatcatctcaatgttaatacctgcctgggccagagtagagaagaacatgcctgcacatccgaccatggccttcattctcgttccgatcagcgacacaatggtcatgcctcgcttcacatctactgtgccgtactttcgcagctcctccacagcctgcttgaggttggagtcgggagcatggaaggccatcgacacatggacctcagaagtggagatgagatcgacgacaagtttctgctggtcgagagtagcaaagatcttgttcaagaagccatgactcttggttcgcttattcgaatgaacgttgagaacggtgatattcgatttggtagtgacagctgtaggcttcttctcgtcatctacaggagcagacacctggggagtgaatgagccagaggacagagaagttagcgagtcggtggaagaagccagatcctggacattgtttttcgttttcaggttgcccgagccgtcagcagatgggtagatgatagttcctcctcccagggggttttccacgtttttgattcggataggaatatgggccttaatgacctgctccatggtgaaggggtggatgacttcagatccgtagtaggtcaactcagcggcctcctcgggggtaatgatgggcagcagacgggcagtggacacctttctgggatcggcagtgaagacaccatcgacttctttccagatctgaagctccttggcgtccagacccacagccaccagagcagcacacagatcggtgtatcctcgtccgatctggcttaggaggcctcccttgacggggccaaagaaaccggtcaacacgggcaccatgtttcccttgacaggagcagagtccgtgttaggagagccgggaagagtgatgatctcgcccagaacacgtccgaggtccgaatagaacttgggatcttcggcattggtggtggtaactgcgtgagacaggtcaaagtaccgtgcgttaacaccagcgtcccgcataactgcagtcatgtacatgcaactgagcttctccccaatggccatgatggagtcgagtgttcggggggagatctcagaaatgatttcagcagcagccaggattcgcaacagctggtcgcactcgccgttgatgtcggcattgaggttttcgagcagttcggggttcttgacgtcacgcttagcagctgcaagatggtcttctcggatagactcaatgatggggttgtaggcgtctgatccgagaagagcagagtcagcagcagcaataagacgggtggtggttccctcggccttggtggcctctagatggcctccttggccccattccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgag

SEQ ID NO: 16 - DNA, 8843 bp, Artificial Sequence
  plasmid for expression of a chimeric YlLIP2pre- CrLIP1 protein
aagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagcccacaaaaatgaagctctctaccattctcttcaccgcttgtgctaccctcgctgctgctgctcccactgctactctcgctaacggtgacactattactggtctgaacgctattattaacgaggctttcctcggtattcccttcgctgagccccccgttggtaaccttcgattcaaggaccccgttccctactctggttctctcgacggtcagaagttcacttcttacggtccctcttgtatgcagcagaaccctgagggtacttacgaggagaacctgcccaaggctgctctcgacctcgttatgcagtctaaggttttcgaggctgtttctccctcttctgaggactgtctcactattaacgttgttcgaccccccggtactaaggctggtgctaacctgcccgttatgctgtggattttcggtggtggtttcgaggttggtggtacttctactttcccccctgctcagatgattactaagtctattgctatgggtaagcccattattcacgtttctgttaactaccgagtttcttcttggggtttcctggctggtgacgagattaaggctgagggttctgctaacgctggtcttaaggaccagcgactcggtatgcagtgggttgctgacaacattgctgctttcggtggtgaccccactaaggttactattttcggtgagtctgctggttctatgtctgttatgtgtcacattctgtggaacgacggtgacaacacttacaagggtaagcccctcttccgagctggtattatgcagtctggtgctatggttccctctgacgctgttgacggtatttacggtaacgagattttcgacctccttgcttctaacgctggttgtggttctgcttctgacaagctggcttgtctgcgaggtgtttcttctgacactctcgaggacgctactaacaacactcccggtttcctcgcttactcttctctgcgactttcttacctccctcgacccgacggtgttaacattactgacgacatgtacgctctcgttcgagagggtaagtacgctaacattcccgttattattggtgaccagaacgacgagggtactttcttcggtacttcttctctcaacgttactaccgacgctcaggctcgagagtacttcaagcagtctttcgttcacgcttctgacgctgagattgacacccttatgactgcttaccccggtgacattactcagggttctcccttcgacactggtattctgaacgctctcactccccagttcaagcgaatttctgctgttctcggtgaccttggtttcactctcgctcgacgatacttccttaaccactacactggtggtactaagtactctttcctctctaagcagctctctggtcttcccgttctgggtactttccactctaacgacattgttttccaggactacctcctgggttctggttctctgatttacaacaacgctttcattgctttcgctactgacctcgaccccaacactgctggtctgctcgttaagtggcccgagtacacttcttcttctcagtctggtaacaacctgatgatgattaacgctctgggtctgtacactggtaaggacaactttcgaactgctggttacgacgctctcttctctaaccctccctctttcttcgtttgaacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcatctagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagg

SEQ ID NO: 17 - DNA, 13802 bp, Artificial Sequence
  plasmid for expression of Cas9; LIP2, LIP3 and LIP8 targeting guide RNAs
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatggatttgctgatgagtccgtgaggacgaaacgagtaagctcgtctggaggcatgatcaacagcggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgttggaccccgtcttcaattacacttcccaactgggaacacccctctttatcgacccattttaggtaatttaccctagcccattgtctccataaggaatattaccctaacccacagtccagggtgcccaggtccttctttggccaaattttaacttcggtcctatggcacagcggtagcgcgtgagattgcaaatcttaaggtcccgagttcgaatctcggtgggacctagttgaaaaatacctctaatgcgccgatggtttagtggtaaaatccatcgttgccatcgatgggcccccggttcgattccgggtcggcgcaggttgacgtacagcaggctgaacgaggatgttttagagctagaaatagcaagttaaaataaggctagtccgttatcaacttgaaaaagtggcaccgagtcggtgcttttttttttcgatcggccccagttgcaaaagttgacacaactctagatctgcttccaaatatagaatcataacaagggttagggtgtgattatataatattggtcttaattgatgtgctagggctttaaaagttggttaaaataacgctctaatgcctttttaatatattgtctttttcaaaatctcaaatcggacacttcttcgtgtatgagactccattttttggctccgtcacgtgatatgtattatcagctatagtggtgtaaacaaagttttttactagctgtaatggcattttgtcggagtggtaaatcgccttcttgttgtgcgttcgagttctggactctgcactgggctactttgaaaaatacctctaatgcgccgatggtttagtggtaaaatccatcgttgccatcgatgggcccccggttcgattccgggtcggcgcaggttgacgttcactcctcagcctcccaaggttttagagctagaaatagcaagttaaaataaggctagtccgttatcaacttgaaaaagtggcaccgagtcggtgcttttttttttcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 18 - DNA, 13246 bp, Artificial Sequence
  plasmid for expression of Cas9; LIP4 targeting guide RNA
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgacgagactgatgagtccgtgaggacgaaacgagtaagctcgtcggtggcctggattcgagtgggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgttggaccccgtcttcaattacacttcccaactgggaacacccctctttatcgacccattttaggtaatttaccctagcccattgtctccataaggaatattaccctaacccacagtccagggtgcccaggtccttctttggccaaattttaacttcggtcctatggcacagcggtagcgcgtgagattgcaaatcttaaggtcccgagttcgaatctcggtgggacctagttgaaaaatacctctaatgcgccgatggtttagtggtaaaatccatcgttgccatcgatgggcccccggttcgattccgggtcggcgcaggttgacgtttacacccactctatcggaggttttagagctagaaatagcaagttaaaataaggctagtccgttatcaacttgaaaaagtggcaccgagtcggtgcttttttttttcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 19 - DNA, 20 bp, Artificial Sequence
  LIP8 CRISPR targeting sequence
acagcaggctgaacgaggat

SEQ ID NO: 20 - DNA, 20 bp, Artificial Sequence
  LIP2 CRISPR targeting sequence
tggaggcatgatcaacagcg

SEQ ID NO: 21 - DNA, 20 bp, Artificial Sequence
  LIP3 CRISPR targeting sequence
tcactcctcagcctcccaag

SEQ ID NO: 22 - DNA, 20 bp, Artificial Sequence
  LIP4 CRISPR targeting sequence
ggtggcctggattcgagtgg

SEQ ID NO: 23 - DNA, 20 bp, Artificial Sequence
  LIP4 CRISPR targeting sequence
ttacacccactctatcggag

SEQ ID NO: 24 - protein, 551 aa, Artificial Sequence
  chimeric protein
MKLSTILFTACATLAAAAPTATLANGDTITGLNAIINEAFLGIPFAEPPVGNLRFKDPVPYSGSLDGQKFTSYGPSCMQQNPEGTYEENLPKAALDLVMQSKVFEAVSPSSEDCLTINVVRPPGTKAGANLPVMLWIFGGGFEVGGTSTFPPAQMITKSIAMGKPIIHVSVNYRVSSWGFLAGDEIKAEGSANAGLKDQRLGMQWVADNIAAFGGDPTKVTIFGESAGSMSVMCHILWNDGDNTYKGKPLFRAGIMQSGAMVPSDAVDGIYGNEIFDLLASNAGCGSASDKLACLRGVSSDTLEDATNNTPGFLAYSSLRLSYLPRPDGVNITDDMYALVREGKYANIPVIIGDQNDEGTFFGTSSLNVTTDAQAREYFKQSFVHASDAEIDTLMTAYPGDITQGSPFDTGILNALTPQFKRISAVLGDLGFTLARRYFLNHYTGGTKYSFLSKQLSGLPVLGTFHSNDIVFQDYLLGSGSLIYNNAFIAFATDLDPNTAGLLVKWPEYTSSSQSGNNLMMINALGLYTGKDNFRTAGYDALFSNPPSFFV

(19) United States
(12) Patent Application Publication
(10) Pub. No.: US 2023/0265443 A1
(43) Pub. Date: Aug. 24, 2023

(54) REDUCTION OF FATTY ACID RETINYL ESTER FORMATION

(71) Applicant: DSM IP ASSETS B.V., Heerlen (NL)

(72) Inventors: Peter Louis HOUSTON, Kaiseraugst (CH); Valmik Kanubhai VYAS, Kaiseraugst (CH); René Marcel DE JONG, Kaiseraugst (CH); Anna SYMBOR-NAGRABSKA, Kaiseraugst (CH); Ethan LAM, Kaiseraugst (CH)

(21) Appl. No.: 18/017,819

(22) PCT Filed: Jul. 22, 2021
(86) PCT No.: PCT/EP2021/070566

§ 371 Date: Jan. 24, 2023

(30) Foreign Application Priority Data
Jul. 27, 2020 (CH) 00929/20

(51) Int. Cl. C12N 15/81; C12N 1/16; C12P 23/00; C12N 9/20

(52) U.S. Cl. CPC C12N 15/815; C12N 1/16; C12P 23/00; C12N 9/20; C12N 2500/36; C12Y 301/01003

(57) ABSTRACT

The present invention is related to a novel process for production of retinyl acetate in a host cell, particularly oleaginous yeast such as e.g. Yarrowia, growing on triglyceride oils, such as e.g. vegetable oil, wherein the host cell exhibits modified lipase activity in such a way that conversion of retinol into fatty acid retinyl esters (FAREs) is reduced or abolished. Such process is especially useful in a biotechnological process for production of vitamin A.

The present invention is related to a novel process for production of retinyl acetate in a host cell, particularly oleaginous yeast such as e.g. Yarrowia, growing on triglyceride oils, such as e.g. vegetable oil, wherein the host cell exhibits modified lipase activity in such a way that conversion of retinol into fatty acid retinyl esters (FAREs) is reduced or abolished. Such process is especially useful in a biotechnological process for production of vitamin A.

Current chemical production methods for retinoids, including vitamin A and precursors thereof, have some undesirable characteristics such as e.g. high-energy consumption, complicated purification steps and/or undesirable by-products. Therefore, over the past decades, other approaches to manufacture retinoids, including vitamin A and precursors thereof, comprising microbial conversion steps have been investigated, which would lead to more economical as well as ecological vitamin A production.

In general, the biological systems that produce retinoids are industrially intractable and/or produce the compounds at such low levels that commercial scale isolation is not practical. Besides instability of intermediates, one of the main limiting factors is the relatively high production of by-products, such as e.g. fatty acid retinyl esters (FAREs), generated via enzymatic conversion of retinol, particularly using oleaginous host cells grown on triglycerides, such as e.g. vegetable oils.

Whereas instability could be solved via expression of highly specific acetylating enzymes (ATFs) in the host cell resulting in increased accumulation of retinyl acetate (see e.g. WO2020/141168), a relatively high percentage of retinol is still “lost” for vitamin A production, i.e. converted into undesired side-products including FARES, that are difficult to purify and do not form a crystal, but instead form a wax. Thus, high concentrations of FAREs limit large scale, industrial production of pure products.

Thus, it is an ongoing task to increase the percentage of retinyl acetate and to reduce FARE formation in bioproduction of vitamin A using a host cell, particularly oleaginous host cells such as Yarrowia growing on triglycerides.

Surprisingly, we now found that modification of the endogenous lipase activity in the host cell, particularly oleaginous yeast, such as e.g. retinyl acetate-producing Yarrowia as defined herein growing on triglycerides, such as e.g. vegetable oil, including modification, particularly blockage, of certain endogenous enzymes involved in pre-digestion of vegetable oil into glycerol and fatty acids with simultaneous addition of certain exogenous or heterologous lipase activity, e.g. fungal lipase activities, particularly lipase activity from oleaginous yeast, such as Candida species, particularly Candida rugosa, could lead to reduced or abolished FARE formation and optionally furthermore increase in the percentage of retinyl acetate, compared to a process using the respective non-modified host cell.

Particularly, the present invention is directed to a retinoid-producing host cell capable of retinyl acetate formation, such as a fungal host cell, preferably oleaginous yeast cell such as e.g. Yarrowia, comprising:
(1) one or more genetic modification(s), i.e. reduction or abolishment, preferably abolishment, of certain endogenous genes encoding enzymes involved in pre-digestion of vegetable oil into glycerol and fatty acids, particularly including e.g. genes encoding endogenous lipases and/or esterases, including but not limited to modification in the activity of an endogenous gene encoding enzymes with activity equivalent to Yarrowia LIP2 and/or LIP3 and/or LIP8 and/or LIP4, such as polypeptides with at least about 50%, such as 60, 70, 80, 90, 95, 98, or 100% identity to SEQ ID NO:1, 3, 5, 7, or combinations thereof, wherein SEQ ID NO:1 corresponds to LIP2 obtainable from Yarrowia lipolytica, SEQ ID NO:3 corresponds to LIP3 obtainable from Yarrowia lipolytica, SEQ ID NO:5 corresponds to LIP8 obtainable from Yarrowia lipolytica, SEQ ID NO:7 corresponds to LIP4 obtainable from Yarrowia lipolytica; and
(2) addition of a heterologous enzyme with lipase activity, particularly fungal lipase activity, particularly from Candida species, such as e.g. Candida rugosa lipase (CrLIP) activity, including but not limited to enzymes with activity equivalent to CrLIP1, such as polypeptides with at least about 75% identity, such as e.g. about 80, 85, 90, 95, 98 or even 100% identity to SEQ ID NO:9.

As used herein, a “host cell” refers to a retinoid-producing host cell, particularly retinyl acetate-producing host cell, particularly oleaginous yeast, comprising two or more (genetic) modification(s) as described herein, such as modifications in the lipase activity, wherein one modification comprises genetic modification, such as e.g. reducing/abolishing activity of endogenous genes encoding enzymes with lipase activities, such as comprising activities equivalent to/corresponding to Yarrowia lipolytica LIP2 and/or LIP3 and/or LIP4 and/or LIP8 activity, including but not limited to deletion of the respective genes, and wherein one modification comprises addition of a heterologous lipase activity, such as e.g. via a further genetic modification of the host cell, i.e. introduction of a heterologous gene/polynucleotide encoding the heterologous lipase, such as activities equivalent to/corresponding to fungal lipases, particularly lipase activity from Candida species, such as e.g. Candida rugosa CrLIP1 activity, particularly lipase originated from Candida species, preferably Candida rugosa, more preferably CrLIP1, or via addition of the heterologous lipase, i.e. administering an effective amount of the heterologous enzyme during the fermentation of the host cell. A host cell comprising the above-described modifications is also referred to as “modified host cell”. The terms “retinoid-producing host cell capable of retinyl acetate formation” and “retinyl acetate-producing host cell” are used interchangeably herein.

As used herein, a “wild-type host cell” means the respective host cell which is wild-type, i.e. non-modified, with respect to the above-mentioned lipase activity modifications. Thus, in a wild-type host cell the corresponding endogenous enzymes as defined herein are (still) expressed and active in vivo and no exogenous lipase activity as defined herein has been added.

As used herein, an enzyme is “expressed and active in vivo” if mRNA encoding for the protein can be detected by Northern blotting and/or protein is detected by mass spectrometry. With regards to the exogenous lipase activity as defined herein it means ability to improve triglyceride utilization of a host cell comprising modification of endogenous lipase activities as defined herein.

Suitable host cells are selected from retinoid-producing host cells, particularly retinyl acetate-producing host cells, wherein retinyl acetate is formed via enzymatic conversion of retinol catalyzed by acetylating enzymes (ATFs), e.g. fungal host cells including oleaginous yeast cells, such as e.g. Rhodosporidium, Lipomyces or Yarrowia, preferably Yarrowia, more preferably Yarrowia lipolytica, comprising a modification in the lipase activity as defined herein.

In one aspect, the present invention is directed to a modified host cell as defined herein capable of retinyl acetate formation as well as to the use of such modified host cell growing on triglycerides, such as e.g. vegetable oil, in a retinyl acetate production process, wherein formation of FARE is reduced during fermentation compared to the formation of FAREs using the respective non-modified/wild-type host cell, such as particularly reduced by at least about 50 to 80% compared to fermentation of a non-modified host cell as defined herein growing on triglycerides. According to some preferred embodiments as described herein, the formation of FARE could be even abolished, i.e. a 100% reduction as compared to the use of the respective non-modified host cell growing on triglycerides, wherein the percentage of FARE is based on total retinoids.

As used herein, the term “endogenous genes encoding enzymes involved in pre-digestion of vegetable oil into glycerol and fatty acids” refers to endogenous enzymes with lipase and/or esterase activity. The term “lipase” is used interchangeably herein with the term “esterase” or “enzyme having lipase and/or esterase activity”. It refers to enzymes involved in pre-digestion of triglyceride oils such as e.g. vegetable oil into glycerol and fatty acids that are normally expressed in oleaginous host cells. Suitable enzymes to be modified in a host cell as defined herein might be selected from endogenous enzymes belonging to EC class 3.1.1.-, including, but not limited to one or more enzyme(s) with activities corresponding to Yarrowia LIP2 and/or LIP3 and/or LIP8 and/or LIP4 activities, including one or more enzyme(s) with at least about 50%, such as 60, 70, 80, 90, 95, 98, or 100% identity to SEQ ID NO:1, 3, 5, 7, or combinations thereof.

As used herein, an enzyme having “activity corresponding/being equivalent to the respective LIP activity in Yarrowia” includes not only the genes originating from Yarrowia, e.g. Yarrowia lipolytica, such as e.g. Yarrowia LIP2, LIP3, LIP8, LIP4 or combinations thereof, but also includes enzymes having equivalent enzymatic activity but are originated from another source organism, particularly retinyl acetate-producing oleaginous host cell, wherein a modification of such equivalent endogenous lipase genes would lead to reduction of FARE formation and/or an increase in retinol to retinyl acetate conversion as defined herein.

In one embodiment, the modified host cell as defined herein, such as modified retinyl acetate-producing oleaginous host cell, comprises a modification in a polypeptide with at least about 50%, such as 60, 70, 80, 90, 95, 98, or 100% identity to SEQ ID NO:5, including but not limited to LIP8 obtainable from Yarrowia lipolytica, wherein the activity of said polypeptide is reduced or abolished, preferably abolished, including reduction or abolishment of gene expression, wherein the use of such modified host cell in a fermentation in the presence of triglycerides, such as e.g. vegetable corn oil, as carbon source results in reduced FARE formation from conversion of retinol as defined herein, with optionally further increase in percentage of retinyl acetate from conversion of retinol, such as e.g. an increase in the range of about 10-30% based on total retinoids compared to the use of the respective non-modified host cell. Particularly, the (modified) host cell is selected from Yarrowia, such as Yarrowia lipolytica, wherein the activity of LIP8 according to SEQ ID NO:5, including a polypeptide encoded by a polynucleotide according to SEQ ID NO:6, is reduced or abolished, preferably abolished, leading to a reduction of at least about 50 to 80% FARE formation based on total retinoids compared to FARE formation using the respective non-modified host cell growing on triglycerides, such as e.g. vegetable oil. Reduction or abolishment of LIP8 or a corresponding enzyme from another oleaginous yeast as defined herein might be combined with reduction or abolishment, particularly abolishment, of further endogenous enzymes including but not limited to enzymes with activities equivalent to Yarrowia LIP2 and/or LIP3 and/or LIP4 activities, including enzymes with at least about 50% identity to a sequence selected from the group consisting of SEQ ID NO:1, 3, 7 and combinations thereof. Particularly preferred is a (modified) host cell, wherein the endogenous enzyme activity corresponding to Yarrowia LIP2, LIP3, LIP8 and optionally together with LIP4 is reduced or abolished, particularly abolished, such as e.g. the corresponding genes are inactive, including but not limited to deletion of the respective genes.

As used herein, the term “addition of an exogenous or heterologous lipase activity” refers to addition of enzymes with lipase activity that are not encoded by endogenous genes of the host cell. Suitable enzymes might be selected from enzymes belonging to EC class 3.1.1.-, including, but not limited to enzymes commercially available, as e.g. available from Novozymes, DSM, Amano, Sigma, Creative Enzymes or other suppliers, such as e.g. heterologous fungal enzymes originated from Candida or Rhizopus species, particularly from C. rugosa, C. cylindracea, R. niveus or R. oryzae. The term “addition” means either in vitro addition, i.e. the administration of such heterologous enzyme, such as e.g. an aqueous solution comprising a mix of one or more enzymes, in an effective amount during the fermentation of the host cell, or in vivo expression, i.e. addition/introduction of a gene/polynucleotide encoding said heterologous enzyme into the host cell—i.e. the host cell is transformed with a polynucleotide encoding said heterologous enzyme as defined herein—such that the respective heterologous enzyme having lipase activity is expressed and active in vivo. Preferably, the host cell is transformed with a heterologous polynucleotide expressing Candida rugosa lipase, such as e.g. an enzyme with activity corresponding to CrLIP1, including an enzyme with at least about 75% identity, such as e.g. about 80, 85, 90, 95, 98 or even 100% identity to SEQ ID NO:9.

As used herein, an enzyme having “activity corresponding/being equivalent to the respective LIP activity in Candida” includes not only the genes originating from Candida, e.g. Candida rugosa, such as e.g. CrLIP1, but also includes enzymes having equivalent enzymatic activity but are originated from another source organism, particularly fungal origin, wherein the addition of such an exogenous or heterologous enzyme activity would lead to reduction of FARE formation and/or an increase in retinol to retinyl acetate conversion as defined herein.

In one preferred embodiment, the heterologous enzyme is expressed in vivo, i.e. the corresponding polynucleotide encoding the heterologous lipase with at least about 75% identity to SEQ ID NO:9 is introduced and expressed in the host cell as defined herein leading to reduction of FARE in the range of at least about 50 to 80%, such as e.g. at least about 50, 55, 60, 65, 70, 75, 80, 85, 90, 95 or even up to 100% compared to a retinyl acetate production process using a non-modified host cell as defined herein.

According to a further preferred embodiment, the host cell as defined herein comprises and expresses a (genetically) modified heterologous lipase, e.g. lipase with activity equivalent to CrLIP1, such as Candida lipase comprising one or more modification(s), such as amino acid substitution(s), in a sequence with at least about 75% identity to SEQ ID NO:9, said one or more amino acid substitution(s) being located at position(s) corresponding to amino acid residue(s) selected from the group consisting of position 296, 344, 345, 448 and combinations thereof, in the polypeptide according to SEQ ID NO:9, preferably comprising one or more amino acid substitution(s) on positions corresponding to amino acid residue(s) 296, 344, 448, or combinations thereof in SEQ ID NO:9.

In one particular embodiment, the (genetically) modified heterologous lipase added to and expressed in vivo in the (modified) host cell as defined herein comprises one or more amino acid substitution(s), wherein the amino acid substitution at a position corresponding to residue 296 in the polypeptide according to SEQ ID NO:9 leads to alanine, valine, isoleucine, serine, asparagine or glutamine, preferably alanine or valine, such as e.g. an amino acid substitution selected from the group consisting of F296A, F296V, F296I, F296S, F296N, and F296Q, wherein substitution of phenylalanine on a residue corresponding to amino acid position F296 in SEQ ID NO:9 might lead to reduction of FARE in the range of about 80 to 100%, i.e. abolishment of FARE formation in a retinyl acetate production process using a modified host cell as defined herein growing on triglycerides and as compared to the respective process using a non-modified host cell.

In one particular embodiment, the (genetically) modified heterologous lipase added to and expressed in vivo in the (modified) host cell as defined herein comprises one or more amino acid substitution(s), wherein the amino acid substitution at a position corresponding to residue 344 in the polypeptide according to SEQ ID NO:9 leads to isoleucine or tryptophan, such as e.g. an amino acid substitution selected from the group consisting of F344I or F344W.

In one particular embodiment, the (genetically) modified heterologous lipase added to and expressed in vivo in the (modified) host cell as defined herein comprises one or more amino acid substitution(s), wherein the amino acid substitution at a position corresponding to residue 345 in the polypeptide according to SEQ ID NO:9 leads to leucine, such as e.g. an amino acid substitution selected from the group consisting of F345L, wherein substitution of phenylalanine on a residue corresponding to amino acid position F345 in SEQ ID NO:9 might lead to reduction of FARE in the range of about 80 to 100%, i.e. abolishment of FARE formation in a retinyl acetate production process using a modified host cell as defined herein growing on triglycerides and as compared to the respective process using a non-modified host cell.

In one particular embodiment, the (genetically) modified heterologous lipase added to and expressed in vivo in the (modified) host cell as defined herein comprises one or more amino acid substitution(s), wherein the amino acid substitution at a position corresponding to residue 448 in the polypeptide according to SEQ ID NO:9 leads to histidine, alanine or tryptophan, such as e.g. an amino acid substitution selected from the group consisting of F448H, F448A or F448W, wherein substitution of phenylalanine on a residue corresponding to amino acid position F448 in SEQ ID NO:9 might lead to reduction of FARE in the range of about 80 to 100% in a retinyl acetate production process using a modified host cell as defined herein growing on triglycerides and as compared to the respective process using a non-modified host cell.

Preferably, the host cell expresses a modified heterologous lipase comprising one or more amino acid substitution(s) on positions corresponding to amino acid residue 296, 344, 345, and/or 448, wherein the substitution on a position corresponding to residue 296, e.g. F296A, F296V, F296I, F296S, F296N, or F296Q is optionally combined with one or more substitution(s) at a position corresponding to residue 344, particularly F344I or F344W, and/or substitution at a position corresponding to residue 345, particularly F345L, and/or substitution at a position corresponding to residue 448, particularly F448H, F448A or F448W, most preferably two amino acid substitutions selected from e.g. F296A_F344W or F296V_F448A, in combination with modification of endogenous lipase activity, such as modification in enzyme activity equivalent to Yarrowia LIP2 and/or LIP3 and/or LIP8 and/or LIP4, i.e. said endogenous enzyme activity being reduced or abolished, particularly wherein the activity of the corresponding endogenous genes are reduced or abolished, including but not limited to deletion of the corresponding genes, more preferably wherein a gene encoding endogenous LIP8 is inactivated, such as e.g. deleted, or wherein genes encoding endogenous LIP2, LIP3 and LIP8 are inactivated, such as e.g. deleted, or wherein genes encoding endogenous LIP2, LIP3, LIP8 and LIP4 are inactivated, such as e.g. deleted, wherein inactivated means that the corresponding endogenous lipase activity cannot be detected (any more) in the host cell.

In one particular embodiment, the present invention is directed to a modified host cell as defined herein capable of retinyl acetate formation as well as to the use of such modified host cell in a retinyl acetate production process, wherein the retinol to retinyl acetate conversion is increased, particularly wherein the percentage of retinyl acetate based on total retinoids is increased by at least about 10 to 30% compared to percentage of retinyl acetate using the respective non-modified/wild-type host cell growing on triglycerides, such as e.g. vegetable oil.

As used herein, “activity” of an enzyme, particularly activity of lipases or esterases as defined herein, is defined as “specific activity” i.e. its catalytic activity, i.e. its ability to catalyze formation of a product from a given substrate, such as e.g. the formation of retinyl fatty esters. An enzyme, e.g. a lipase or esterase, is active, if it performs its catalytic activity in vivo, i.e. within the host cell as defined herein or within a system in the presence of a suitable substrate. The skilled person knows how to measure enzyme activity, in particular activity of lipases as defined herein, including but not limited to enzyme with activities corresponding to Yarrowia LIP2 and/or LIP3 and/or LIP8 and/or LIP4 activity. Analytical methods to evaluate the capability of lipases/esterases as defined herein involved in formation of retinyl fatty esters are known in the art and include measurement via HPLC and the like. With regards to activity of LIP2, LIP3, LIP8, LIP4 and/or the heterologous lipase enzymes, e.g. fungal lipases such as e.g. Candida lipases, particularly CrLIP1, as defined herein, the skilled person might measure the formation of retinyl fatty esters from conversion of retinol in comparison to the formation of retinyl acetate from conversion of retinol, both measured with a modified and the respective wild-type host cell.

As used herein, an enzyme, particularly a lipase or esterase as defined herein, having “reduced or abolished” activity means a decrease in its specific activity, i.e. reduced/abolished ability to catalyze formation of a product from a given substrate, such as conversion of triglycerides, such as e.g. vegetable oil, preferably corn oil, into glycerol and fatty acids during fermentation, including reduced or abolished activity of the respective (endogenous) gene encoding such lipases or esterases. A reduction by 100% is referred herein as abolishment of enzyme activity, achievable e.g. via deletion, insertions, frameshift mutations, missense mutations or premature stop-codons in the endogenous gene encoding said enzyme or blocking of the expression and/or activity of said endogenous gene(s) with known methods, particularly combined with addition of exogenous or heterologous lipase activity as defined herein.

The (modified) host cell according to the present invention might be originated from Yarrowia lipolytica as disclosed in WO2019/058001 or WO2019/057999, thus further genetically modified, wherein the formation of retinyl acetate from beta-carotene is optimized via heterologous expression of beta-carotene oxidases (BCO), retinol dehydrogenase (RDH) and/or acetyl-transferases (ATF). Particularly, a modified host cell as defined herein might be expressing a BCO originated from Drosophila melanogaster or Danio rerio, RDH originated from Fusarium fujikuroi, and fungal ATF, such as e.g. ATF originated from Lachancea or Saccharomyces. To enhance the conversion of beta-carotene into retinal into retinol into retinyl acetate in a process as defined herein, said enzymes might comprise one or more mutations leading to improved acetylation of retinol into retinyl acetate.

Introduction of modification(s) in the retinoid-producing host cell capable of retinyl acetate formation as defined herein in order to produce less or no copies of genes and/or proteins, such as endogenous lipases or esterases and respective genes as defined herein, including generation of modified suitable host cell capable of retinyl acetate formation as defined herein with reduced/abolished activity in endogenous enzymes corresponding to Yarrowia LIP2 and/or LIP3 and/or LIP8 and/or LIP4 may include the use of weak promoters, or the introduction of one or more mutations) (e.g. insertion, deletion/knocking-out or point/frameshift/missense mutation, premature stop-codons) of (parts of) the respective enzymes (as described herein), in particular its regulatory elements, leading to reduction/abolishment of said enzyme activity, such as e.g. inactivation via in vivo mutagenesis, for example by mutation of the catalytic residues or by making mutations or deletions that interfere with protein folding or pre- or pro-sequence cleavage needed to activate the lipase/esterase upon secretion by the host cell. The skilled person knows how to genetically manipulate or modify a host cell as defined herein resulting in reduction/abolishment of such endogenous activity, e.g. lipase or esterase activity, as defined herein. These genetic manipulations include, but are not limited to, e.g. gene replacement, gene amplification, gene disruption, transfection, transformation using plasmids, viruses, or other vectors. An example of such a genetic manipulation may for instance affect the interaction with DNA that is mediated by the N-terminal region of enzymes as defined herein or interaction with other effector molecules. In particular, modifications leading to reduced/abolished specific enzyme activity may be carried out in functional, such as functional for the catalytic activity, parts of the proteins.

Furthermore, reduction/abolishment of enzyme specific activity might be achieved by contacting said enzymes with specific inhibitors or other substances that specifically interact with them. In order to identify such inhibitors, the respective enzymes, such as e.g. certain endogenous lipases as defined herein, may be expressed and tested for activity in the presence of compounds suspected to inhibit their activity.

As used herein, “deletion” of a gene leading to abolishment of gene activity includes all mutations in the nucleic acid sequence that can result in an allele of diminished function, including, but not limited to deletions, insertions, frameshift mutations, missense mutations, and premature stop codons, wherein deleted means that the corresponding gene/protein activity, such as particularly endogenous lipase activity, cannot be detected (anymore) in the host cell.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

Thus, the present invention furthermore includes a process for identification of endogenous enzymes to be modified, such as e.g. via reduction or abolishment of the specific enzyme activity, including lipases/esterases with activities corresponding to Yarrowia LIP2 and/or LIP3 and/or LIP8 and/or LIP4, comprising the step of over-expressing the respective endogenous genes one by one in a suitable host cell, such as e.g. retinyl-acetate-producing host cell, to see if that amplifies a negative effect, like decreasing the percentage of retinyl acetate. Subsequently, one can reduce/abolish, e.g. inactivate the corresponding genes such as e.g. via deletion, the activity of those enzymes for which this overexpression leads to reduction in retinyl acetate and/or increases FARE during fermentation of said host cell, and picking the clones with reduced FARE formation.

A particular embodiment is directed to a process for production of retinyl acetate in a modified host cell as defined herein comprising identification of suitable endogenous lipases to be modified as defined herein comprising the steps of:
pre-digestion of vegetable oil into glycerol and fatty acids,
(2) selection of endogenous lipase or esterase enzymes based on sequence homology of at least about 50%, such as e.g. 60, 70, 80, 90, 95, 98 or 100% to SEQ ID NO:1, 3, 5, 7,
(3) overexpression of selected genes and comparison of FARE formation and/or retinyl acetate percentage based on total retinoids,
(4) selection of genes, wherein overexpression had a positive impact on FARE formation and/or negative impact on retinyl acetate percentage in the retinoid mix, and
(5) reduction or abolishment, e.g. inactivation, such as e.g. via deletion, of selected genes which upon overexpression had a positive impact on FARE formation and/or negative impact on retinyl acetate formation.

Preferably, such process furthermore comprises the steps of adding a suitable heterologous lipase as defined herein, particularly wherein the heterologous lipase comprises one or more amino acid substitution(s) as defined herein.

According to one specific aspect of the present invention, the modified host cell as defined herein might be used in a process for reducing the formation of FAREs in vitamin A fermentation process and optionally increasing the percentage of retinyl acetate based on total retinoids and preferably retinoids produced by the modified host cell in a fermentation in the presence of triglycerides as carbon source. The modified host cell as defined herein with reduction or abolishment of endogenous lipase activity as defined herein might comprise further modifications, including the introduction (and expression) of (host-optimized) heterologous polynucleotides, such as e.g. the heterologous lipase enzymes as defined herein. The skilled person knows how to generate such modified polynucleotides. It is understood that such host-optimized nucleic acid molecules as well as molecules comprising so-called silent mutations are included by the present invention as long as they still result in modified host cells carrying modified lipase/esterase activity as defined herein.

The terms “sequence identity”, “% identity” or “sequence homology” are used interchangeable herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity. With regards to enzymes originated from plants, the skilled person knows plant-derived enzymes might contain a chloroplast targeting signal which is to be cleaved via specific enzymes, such as e.g. chloroplast processing enzymes (CPEs).

In one embodiment, the host cell to be used in the present invention might express further enzymes, such as heterologous ATF, particularly fungal ATF, comprising a highly conserved partial amino acid sequence of at least 7 amino acid residues selected from [NDEHCS]-H-x(3)-D-[GA] (motifs are in Prosite syntax, as defined in https://prosite.expasy.org/scanprosite/scanprosite_doc.html, wherein “x” denotes an arbitrary amino acid and with the central histidine being part of the enzyme's binding pocket, preferably wherein the 7 amino acid motif is selected from [NDE]-H-x(3)-D-[GA], more preferably selected from [ND]-H-x(3)-D-[GA], most preferably selected from N-H-x(3)-D-[GA] corresponding to position N218 to G224 in the polypeptide according to SEQ ID NO:1 in WO2020/141168. Examples of such enzymes might be particularly selected from L. mirantina, L. fermentati, S. bayanus, or W. anomalus, such as e.g. LmATF1 according to SEQ ID NO:1 in WO2020/141168, SbATF1, LffATF1, LfATF1, Wa1ATF1 or Wa3ATF1 as disclosed in WO2019/058001, more preferably said ATFs comprising one or more amino acid substitution(s) in a sequence with at least about 20%, such as e.g. 25, 30, 35, 40, 45, 50, 55, 60, 65, 70, 75, 80, 85, 90, 92, 95, 97, 98, 99% or up to 100% identity to SEQ ID NO:1 in WO2020/141168, wherein the one or more amino acid substitution(s) are located at position(s) corresponding to amino acid residue(s) selected from the group consisting of position 68, 69, 72, 73, 171, 174, 176, 178, 291, 292, 294, 301, 307, 308, 296, 312, 320, 322, 334, 362, 405, 407, 409, 480, 483, 484, 490, 492, 520, 521, 522, 524, 525, 526 and combinations thereof and as particularly exemplified in Table 4 of WO2020/141168, most preferably comprising one or more amino acid substitution(s) on positions corresponding to amino acid residue(s) 69, 407, 409, 480, 484, and combinations thereof in SEQ ID NO:1 in WO2020/141168.

In one particular embodiment, the modified host cell to be used for the process according to the present invention comprises an amino acid substitution at a position corresponding to residue 69 in the ATF according to SEQ ID NO:1 in WO2020/141168 leading to asparagine, serine or alanine at said residue, such as e.g. via substitution of histidine by asparagine (H69N), serine (H695) or alanine (H69A), with preference for H69A. Said modified enzyme might be originated from yeast, such as e.g. L. mirantina, L. fermentati, W. anomalus or S. bayanus, preferably from L. mirantina, optionally being combined with amino acid substitution at a position corresponding to residue 407 in the ATF according to SEQ ID NO:1 in WO2020/141168 leading to isoleucine at said residue, such as e.g. via substitution of valine by isoleucine (V407I), optionally being combined with an amino acid substitution at a position corresponding to residue 409 in the ATF according to SEQ ID NO:1 in WO2020/141168 leading to alanine at said residue, such as e.g. via substitution of glycine by alanine (G409A), optionally being combined with amino acid substitution at a position corresponding to residue 480 in the ATF according to SEQ ID NO:1 in WO2020/141168 leading to glutamic acid, lysine, methionine, phenylalanine or glutamine at said residue, such as e.g. via substitution of serine by glutamic acid (5480E), lysine (5480L), methionine (5480M), phenylalanine (5480F) or glutamine (5480Q), optionally being combined with amino acid substitution at a position corresponding to residue 484 in the ATF according to SEQ ID NO:1 in WO2020/141168 leading to leucine at said residue, such as e.g. via substitution of isoleucine by leucine (1484L). Said modified enzyme might be originated from yeast, such as e.g. L. mirantina, L. fermentati, W. anomalus or S. bayanus, preferably from L. mirantina. In a most preferred embodiment, the ATF to be used for the process according to the present invention is a modified ATF comprising amino acid substitutions S480Q_G409A_V407I_H69A_I484 L and is obtainable from Lachancea mirantina.

Optionally, the host cell as defined herein, such as e.g. Yarrowia, capable of producing retinyl acetate from conversion of retinol, is expressing further enzymes used for biosynthesis of beta-carotene and/or additionally used for catalyzing conversion of beta-carotene into retinal and/or retinal into retinol. The skilled person knows which genes to be used/expressed for either biosynthesis of beta-carotene and/or bio-conversion of beta-carotene into retinol. Such modified host cell as defined herein further being capable of expressing ATF genes as defined herein and/or further genes required for biosynthesis of vitamin A, is cultured in an aqueous medium supplemented with appropriate nutrients under aerobic or anaerobic conditions and as known by the skilled person for the different host cells, including the presence of triglyceride oils, such as e.g. vegetable oil, particularly corn oil, as carbon source. The cultivation/growth of the host cell may be conducted in batch, fed-batch, semi-continuous or continuous mode. Depending on the host cell, preferably, production of retinoids such as e.g. vitamin A and precursors such as retinal, retinol, retinyl acetate can vary, as it is known to the skilled person. Cultivation and isolation of beta-carotene and retinoid-producing host cells selected from Yarrowia is described in e.g. WO2008/042338.

Carbon sources to be used for the present invention are all suitable triglyceride oils including but not limited to prehydrolysed oils containing free fatty acids like oleic, palmitic, steric or linoleic acid and glycerol, such as e.g. vegetable oil, including but not limited to corn, olive, cottonseed, rapeseed, sesame, canola, safflower, sunflower, soybean, grapeseed, or peanut oil, preferably corn oil.

Fermentation products including retinyl acetate may be harvested from the cultivation at a suitable moment, e.g. when one or more of the nutrients are exhausted. Depending on the host cell, preferably, production of retinoids such as e.g. vitamin A, precursors and/or derivatives thereof such as retinal, retinol, retinyl acetate, particularly retinyl acetate, can vary, as it is known to the skilled person. The retinoids including but not limited to retinol, retinyl acetate, vitamin A might be used as ingredients/formulations in the food, feed, pharma or cosmetic industry.

In a particular embodiment, the present invention is directed to a process for production retinyl acetate with a percentage of FAREs reduced by at least about 50 to 80% based on total retinoids, said process comprising the steps of:
(a) providing a retinoid-producing host cell capable of formation of retinyl acetate,
(b) introduction of one or more modification(s) into the genome of said host cell, such as modification(s) into enzyme(s) belonging to the EC class 3.1.1.- having lipase/esterase activity, such as e.g. reducing/abolishing the enzyme activity including but not limited to deletion of the respective genes, particularly abolishment of lipase activity corresponding to Yarrowia LIP8 and optionally further abolishing enzyme activity corresponding to Yarrowia LIP2 and/or LIP3 and/or LIP4, wherein the modified host cell is growing on triglyceride oils, such as e.g. vegetable corn oil, as carbon source;
(c) adding heterologous lipase activity as defined herein
(d) optionally introduction of further modification(s) comprising expression of one or more copies of (heterologous) enzymes involved in retinol, retinyl acetate and/or vitamin A production as known to a person skilled in the art,
(d) cultivation of such modified host cell under suitable conditions resulting in formation of retinyl acetate, wherein the modified host cell is grown on triglycerides, particularly vegetable oil, as carbon source; and
(e) optionally isolation and/or further purification of retinyl acetate from the cultivation (fermentation) medium.

“Retinoids” or a “retinoid-mix” as used herein include vitamin A, precursors and/or intermediates of vitamin A such as beta-carotene cleavage products also known as apocarotenoids, including but not limited to retinal, retinoic acid, retinol, retinoic methoxide, retinyl acetate, retinyl fatty esters, 4-keto-retinoids, 3 hydroxy-retinoids or combinations thereof. Biosynthesis of retinoids is described in e.g. WO2008/042338. A host cell capable of production of retinoids in e.g. a fermentation process is known as “retinoid-producing host cell”. The genes of the vitamin A pathway and methods to generate retinoid-producing host cells are known in the art (see e.g. WO2019/058000), including but not limited to beta-carotene oxidases, retinol dehydrogenases and/or acetyl transferases. Suitable acetyl transferase enzymes (ATFs) capable of acetylation of retinol into retinyl acetate are disclosed in e.g. WO2019/058001 or WO2020/141168. Suitable beta-carotene oxidases leading to high percentage of trans-retinal are described in e.g. WO2019/057999. A “retinyl-acetate producing host cell” as used herein is expressing suitable ATFs catalyzing the conversion of retinol into retinyl acetate.

The terms “triglycerides” and “triglyceride oils” are used interchangeably herein.

“FARES” or “retinyl fatty esters” as used interchangeably herein includes long chain retinyl esters. These long chain retinyl esters define hydrocarbon esters that consists of at least about 8, such as e.g. 9, 10, 12, 13, 15 or 20 carbon atoms and up to about 26, such as e.g. 25, 22, 21 or less carbon atoms, with preferably up to about 6 unsaturated bonds, such as e.g. 0, 1, 2, 4, 5, 6 unsaturated bonds. Long chain retinyl esters include but are not limited to linoleic acid, oleic acid, or palmitic acid.

“Vitamin A” as used herein may be any chemical form of vitamin A found in aqueous solutions, in solids and formulations, and includes retinol, retinyl acetate and retinyl esters. It also includes retinoic acid, such as for instance undissociated, in its free acid form or dissociated as an anion.

“Retinal” as used herein is known under IUPAC name (2E,4E,6E,8E)-3,7-Dimethyl-9-(2,6,6-trimethylcyclohexen-1-yl)nona-2,4,6,8-tetraenal. It includes both cis- and trans-isoforms, such as e.g. 11-cis retinal, 13-cis retinal, trans-retinal and all-trans retinal. For the purpose of the present invention, the formation of trans-retinal is preferred, which might be generated via the use of stereoselective beta-carotene oxidases, such as described in e.g. WO2019/057999.

“Carotenoids” as used herein include long, 40 carbon conjugated isoprenoid polyenes that are formed in nature by the ligation of two 20 carbon geranylgeranyl pyrophosphate molecules. These include but are not limited to phytoene, lycopene, and carotene, such as e.g. beta-carotene, which can be oxidized on the 4-keto position or 3-hydroxy position to yield canthaxanthin, zeaxanthin, or astaxanthin. Biosynthesis of carotenoids is described in e.g. WO2006/102342. Cells capable of carotenoid production via one or more enzymatic conversion steps leading to carotenoids, particularly to beta-carotene, i.e. wherein the respective polypeptides involved in production of carotenoids are expressed and active in vivo are referred to herein as carotenoid-producing host cells. The genes and methods to generate carotenoid-producing cells are known in the art, see e.g. WO2006/102342. Depending on the carotenoid to be produced, different genes might be involved.

Conversion according to the present invention is defined as specific enzymatic activity, i.e. catalytic activity of enzymes described herein, including but not limited to the enzymatic activity of lipases or esterases, in particular enzymes belonging to the EC class 3.1.1.- involved in conversion of retinol into retinyl fatty esters, beta-carotene oxidases (BCOs), retinol dehydrogenases (RDHs), acetyl transferases (ATFs).

With regards to the present invention, it is understood that organisms, such as e.g. microorganisms, fungi, algae, or plants also include synonyms or basonyms of such species having the same physiological properties, as defined by the International Code of Nomenclature of Prokaryotes or the International Code of Nomenclature for algae, fungi, and plants (Melbourne Code). Thus, for example, strain Lachancea mirantina is a synonym of strain Zygosaccharomyces sp. IFO 11066, originated from Japan.

The following examples are illustrative only and are not intended to limit the scope of the invention in any way. The contents of all references, patent applications, patents, and published patent applications, cited throughout this application are hereby incorporated by reference, in particular WO2020/141168, WO2019/058001, WO2019/057999, WO2008/042338, WO2019/058000, WO2006/102342, WO2017/115322, WO2017/211930.

### EXAMPLES

### Example 1: General Methods and Strains

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

Shake plate assay. Typically, 200 μl of 0.25% yeast extract, 0.5% peptone (0.25×YP) is inoculated with 10 μl of freshly grown Yarrowia and overlaid with 200 μl of mineral oil (Drakeol 5, Penreco, Karns City, Pa., USA). Lipases resuspended in PBS were added to the growth media. The carbon source was 2% corn oil in mineral oil. Transformants were purified by steaking to single colonies, grown in 24 well plates in a shaking incubator (Infors Multitron, 30° C., 800 RPM) in media described above for 4 days at 30° C. The second phase (Drakeol 5) fraction was removed from the shake plate wells and analyzed by UPLC on a normal phase column and/or C4 HPLC, with a photo-diode array detector (see details below).

DNA transformation. Strains were transformed by overnight growth on YPD plate media; 50 μl of cells were scraped from a plate and transformed by incubation in 500 μl with 1 μg transforming DNA, typically linear DNA for integrative transformation, 40% PEG 3550 MW, 100 mM lithium acetate, 50 mM Dithiothreitol, 5 mM Tris-Cl pH 8.0, 0.5 mM EDTA for 30 minutes at 40° C. and plated directly to selective media or in the case of dominant antibiotic marker selection the cells were out grown on YPD liquid media for 4 hours at 30° C. before plating on the selective media. URA3 marker recycling was performed using 5-fluoroorotic acid (FOA). Episomal hygromycin resistance marker (Hyg) plasmids were cured by passage on non-selective media, with identification of Hyg-sensitive colonies by replica plating colonies from non-selective media to hygromycin containing media (100 μg/mL). Selection of the nourseothricin-resistance marker (Nat) was performed on YPD media containing nourseothricin (100 μg/mL).

DNA molecular biology. Plasmids MB9523 containing expression systems for DrBCO, LmATF-S480Q_G409A_V407I_H69A_I484 L, and FfRDH (SEQ ID NO:14) and MB9721 (SEQ ID NO:15) for the expression of a chimeric YlLIP2pre-CrLIP1 protein (SEQ ID NO:16) were synthesized at Genscript (Piscataway, N.J., USA). Both plasmids MB9523 and MB9721 contain the ‘URA3’ for marker selection in Yarrowia lipolytica transformations. For clean gene insertion by random nonhomologous end joining of the gene and marker HindIII/Xbal (MB9721) or Sfil (MB9523), plasmid fragments of interest were purified by gel electrophoresis and Qiagen gel purification column. Clones were verified by sequencing. Typically, genes are synthesized by a synthetic biology at GenScript (Piscataway, N.J.). Plasmids MB9287 and MB9953, containing a Cas9, and guide RNA expression systems to target LIP2, LIP3, and LIP8 in the case of MB9287, and LIP4 in the case of MB9953, were synthesized at Genscript (Piscataway, N.J., USA).

Plasmid list. Plasmid, strains, nucleotide and amino acid sequences that were used are listed in Table 1, 2, 3 and the sequence listing. In general, all non-modified sequences referred to herein are the same as the accession sequence in the database for reference strain CLIB122 (Dujon B, et al, Nature. 2004 Jul. 1; 430(6995):35-44).

TABLE 1
list of plasmids used for construction of the strains for
overexpression or deletion of the respective genes indicated
as “insert”. “LmATF1-mut” refers to Lachancea
mirantina (LmATF1; SEQ ID NO: 13 in WO2019/058001) carrying
aa substitutions S480Q_G409A_V407I_H69A_I484L. “DrBCO”
refers to BCO originated from Danio rerio (see SEQ ID NO:
18 in WO2020/141168); “FfRDH” refers to RDH originated
from Fusarium fujikuroi (see SEQ ID NO: 22 in WO2020/141168).
For more explanation, see text.

| Plasmid | Insert | Marker | SEQ ID NO: |
|---|---|---|---|
| MB7452 | Cas9 | Nat | 14 |
| MB9523 | DrBCO; LmATF1-mut; FfRDH | URA3 | 15 |
| MB9721 | YlLIP2pre-CrLIP1 chimera | URA3 | 16 |
| MB9287 | Cas9; LIP2, LIP3 and LIP8 | Hyg | 17 |
| | targeting guide RNAs | | |
| MB9953 | Cas9; LIP4 targeting guide RNA | Hyg | 18 |

TABLE 2
list of Yarrowia lipolytica strains used. Construction of ML17544 is
described in Table 2 of WO2020/141168. For more details, see text.

| Strain | Description |
|---|---|
| ML18812 | ML17544 transformed with MB9523 |
| ML18743 | ML18812 with MB9287 lip8, lip2, |
| | lip3 deletion |
| ML18743-lip4 | ML18743 with MB9953 lip4 deletion |
| ML18756 | ML18743 cured of URA3 by FOA |
| ML18870 | ML18756 transformed with MB9721 |

TABLE 3
DNA sequences targeted using Cas9 CRISPR for
mutation of lipase genes. “Lip gene” means the
respective lipase gene from Yarrowia lipolytica.
“CRISPR targeting sequence” is the seed sequence
used for Cas9 CRISPR targeting. The respective
guide RNA expression plasmid for LIP8, LIP2
and LIP3 constructs is MB9287, for LIP4 it
is MB9953. For more details, see text.

| | CRISPR targeting | SEQ ID |
|---|---|---|
| Lip gene | sequence | NO: |
| LIP8 | ACAGCAGGCTGAACGAGGAT | 19 |
| LIP2 | TGGAGGCATGATCAACAGCG | 20 |
| LIP3 | TCACTCCTCAGCCTCCCAAG | 21 |
| LIP4 | GGTGGCCTGGATTCGAGTGG | 22 |
| LIP4 | TTACACCCACTCTATCGGAG | 23 |

Retinoid quantification. Analysis of retinoids were carried out with a C4 reverse phase retinoid method (see below) and C18 as described elsewhere (WO2020/141168). The addition of all added intermediates gives the total amount of retinoids.

C4 reverse phase chromatography. For exact determination of discrete retinoids the long run reverse phase system was used. We separated analytes at 230 nm and 325 nm through the Agilent 1290 instrument with YMC Pro C4, 150×3.0 mm 3 μm column (YMC America, Allentown Pa.) stationary phase, and a 5 μl injection loop volume and column and sample tray controlled at 23° C. with gradients described in Table 4B. Analytes were detected at 230 nm and 325 nm and the peaks identity verified with LCMS. The analytes separated as discrete peaks that were assigned according to Table 4A.

TABLE 4A
list of analytes using C4-reverse phase method. The addition
of all added intermediates gives the total amount retinoids.
“RT” means retention time. For more details, see text.

| Intermediates | RT [min] | λ max [nm] |
|---|---|---|
| trans-retinol | 20.21 | 325 |
| cis-retinol | 20.32 | 325 |
| dihydro-retinol | 20.75 | 290 |
| trans-retinal | 20.89 | 380 |
| cis-retinal | 21.02 | 380 |
| trans-retinyl-acetate | 22.15 | 325 |
| cis-retinyl-acetate | 22.35 | 325 |
| dihydro-retinyl acetate | 22.60 | 290 |
| retinyl esters | 26.30 | 325 |

TABLE 4B
UPLC Method Gradient with solvent A: acetonitrile; solvent
B: water; solvent C: water/acetonitrile/methanesulfonic
acid 1000:25:1. For more details, see text.

| Time | | | | Flow |
|---|---|---|---|---|
| [min] | % A | % B | % C | [ml/min] |
| 0 | 5 | 85 | 10 | 0.5 |
| 20 | 98 | 0 | 2 | 0.5 |
| 35 | 98 | 0 | 2 | 0.5 |
| 35.1 | 5 | 85 | 10 | 0.5 |
| 40 | 5 | 85 | 10 | 0.5 |

Method Calibration. Method is calibrated using high purity retinyl acetate received from DSM Nutritional Products, Kaiseraugst, C H. Retinols and retinal are quantitated against retinyl acetate. Dilutions described in Table 4C are prepared as follows. 40 mg of retinyl acetate is weighed into a 100 mL volumetric flask, and dissolved in ethanol, yielding a 400 μg/mL solution. This solution is sonicated as required to ensure dissolution. 5 mL of this 400 μg/mL solution is diluted into 50 mL (1/10 dilution, final concentration 40 μg/mL), 5 mL into 100 mL (1/20 dilution, final concentration 20 μg/mL), 5 mL of 40 μg/mL into 50 mL (1/10 dilution, final concentration 4 μg/mL), 5 mL of 20 μg/mL into 50 mL (1/10 dilution, 2 μg/mL), using 50/50 methanol/methyl tert-butyl ether(MTBE) as the diluent. All dilutions are done in volumetric flasks. Purity of retinyl acetate is determined by further diluting the 400 μg/mL stock solution 100-fold (using a 2 mL volumetric pipet and a 200 mL volumetric flask) in ethanol. Absorbance of this solution at 325 nm using ethanol is taken as the blank, with adjustment of the initial concentration using the equation (Abs*dilution (100)*molecular weight (328.5)/51180=concentration in mg/mL). Because of quick out-maximization of UV absorbance of retinyl acetate, lower concentrations are better.

TABLE 4C
preparation of calibration standards. For more explanation, see text.

| Stock [RA], dilution | Final concentration |
|---|---|
| 20 μg/mL, 1/10 | 2 μg/mL |
| 40 μg/mL, 1/10 | 4 μg/mL |
| 400 μg/mL, 1/20 | 20 μg/mL |
| 400 μg/ml, 1/10 | 40 μg/mL |

Sample preparation. Top second phase layer samples from each strain were diluted at a 25-fold dilution or higher into tetrahydrofuran (THF). Fermentation whole broth was prepared using a 2 mL Precellys (Bertin Corp, Rockville, Md.) tube, add 25 μl of well mixed broth and 975 μl of THF. Precellys 3×15×7500 rpm for two cycles with a freeze at −80° C. for 10 minutes between cycles. Cell debris was spun down via centrifugation for 1 minute at 13000 rpm. These samples were diluted 10-fold in THF.

### Example 2: Deletion of Lipase Genes in Yarrowia lipolytica

Lipase genes (LIP2, LIP3, & LIP8) were disabled in strain ML18812 using modern CRISPR Cas9 methods, using the CRISPR sites indicated in Table 3, to generate strain ML18743. Briefly, strain ML18812 was transformed with MB7452 (SEQ ID NO:14), which contains an expression module for Cas9 and the nourseothricin selection marker. Nourseothricin resistant transformants (selected on YPD with 200 μg/mL nourseothricin) were subsequently transformed with plasmid MB9287, which contains expression sequences for Cas9, and guide RNAs with seed sequences targeting LIP2, LIP3, and LIP8 (shown in Table 3), and the hygromycin resistance marker. Transformants (selected on YPD with 200 μg/mL hygromycin) were screened for mutation by Sanger sequencing with primers flanking the targeted region. Strain ML18743 was found to have inactivating mutations in LIP2, LIP3, and LIP8. Whereas formation of FARE using such strain growing on corn oil could be more or less abolished, the growth rate of such lip deletion strains was very low compared to strain ML18812 (see Table 6).

### Example 3: Addition of Heterologous Lipases in Growth-Deficient LIP2-3-8-Deletion Strains of Yarrowia lipolytica

To correct for the defect of low growth rates (see Ex. 2), commercially available lipases from Candida rugosa (CrLIP Sigma), Candida cylindracea (CcLIP, Creative Enzymes), Rhizopus niveus (Rn LIP, Sigma), and Rhizopus oryzae (RoLIP, Creative enzymes) as well as lipases from DSM (CrLIP1, CrLIP2, CrLIP3, CrLIP4, and CrLIP5) were added to strain ML18743 (comprising lip2-3-8 deletion) inoculated to 0.25xYEP medium with 2% corn oil as the sole carbon source (Table 5).

Commercially produced Cc and Cr lipase preparations (Creative Enzymes and Sigma Aldrich) are typically a mixture of several lipases natively expressed by Candida rugosa/Candida cylindracea. Lipases from DSM were produced by the protocol according to Example 1 in WO2017/115322.

TABLE 5
heterologous lipases added to the fermentation medium.
Either the catalog number or SEQ ID NO: (for lipases
from DSM) are given. For more explanation, see text.

| | | Catalog number/ |
|---|---|---|
| Lipase | Manufacturer | SEQ ID NO: |
| CrLIP | Sigma Aldrich | L1754 |
| CcLIP | Creative Enzymes | DIS-1027 |
| RnLIP | Sigma Aldrich | 62310 |
| RoLIP | Creative Enzymes | DIS-1026 |
| Lipozyme TL | Novozymes | 06-3155 |
| Novacor | Novozymes | 06-3100 |
| Palatase | Novozymes | 06-3118 |
| Resinase | Novozymes | 06-3125 |
| CrLIP1 | DSM | 9 |
| CrLIP2 | DSM | 10 |
| CrLIP3 | DSM | 11 |
| CrLIP4 | DSM | 12 |
| CrLIP5 | DSM | 13 |

While all enzymes were able to break down triglycerides in corn oil to permit growth, only CrLip, CcLip, RnLIP, RoLIP, CrLIP1, CrLIP2, and CrLIP5 were able to do so with minimal FARE production in the lip2 lip3 lip8 retinyl acetate producing strain ML18743. Results are depicted in Table 6.

TABLE 6
retinoid production in strain ML18743 (deletion of LIP2-3-8) compared to wild-
type strain ML18812 (“LIP+”) as control with or without addition of
the respective lipase according to Table 5. “% FARE” is based on total
retinoids produced with addition of each individual lipase. “Total retinoids
(% of LIP+)” is the percentage of retinoids produced compared to total
retinoids in the control without addition of lipases, wherein the total retinoids
obtained with the control are set to 100%. For growth on corn oil,“+++”
indicates growth in the upper quartile, while “+” indicates growth
in the lower quartile. For more explanation, see text.

| | | Total | | | |
|---|---|---|---|---|---|
| | | retinoids | | Growth on | Lipase Units |
| Strain | Lipase | (% of LIP+) | % FARE | corn oil | added/well |
| ML18812 | none | 100 | 88.54 | +++ | 0 |
| ML18743 | none | 4 | 0.00 | + | 0 |
| ML18743 | CrLIP | 51.18 | 5.86 | +++ | 0.28 |
| ML18743 | CcLIP | 192.65 | 15.13 | +++ | 0.08 |
| ML18743 | RnLIP | 56.50 | 0.00 | +++ | 0.06 |
| ML18743 | RoLIP | 53.47 | 3.49 | +++ | 0.2 |
| ML18743 | Lipozyme TL | 225.32 | 91.74 | +++ | 0.4 |
| ML18743 | Novacor | 166.64 | 80.83 | +++ | 0.24 |
| ML18743 | Palatase | 172.10 | 95.70 | +++ | 0.8 |
| ML18743 | Resinase | 166.22 | 95.46 | +++ | 0.2 |
| ML18743 | CrLIP1 | 33.29 | 15.41 | +++ | 0.1376 |
| ML18743 | CrLIP2 | 7.66 | 7.69 | + | 0.7 |
| ML18743 | CrLIP3 | 31.56 | 74.34 | +++ | 0.0428 |
| ML18743 | CrLIP4 | 66.28 | 70.35 | +++ | 0.08 |
| ML18743 | CrLIP5 | 4.26 | 0.00 | + | 0.04 |

From this data, we conclude CrLip1 was able to simultaneously enable growth/retinoid production while minimizing production of FAREs, and most reflected the observed behavior of commercially available lipases, e.g., CrLIP, CcLIP, RoLIP or RnLIP (see Table 6).

### Example 4: Addition of CrLIP1 Mutants in Growth-Deficient LIP2-3-8-Deletion Strains of Yarrowia lipolytica

Several mutant CrLIP1 proteins were produced (for a full description to produce these mutants in Pichia pastoris, see Example 1 and 2 in WO2017/211930), including double mutants and tested in our shake plate assay. Results are shown in Table 7. Several of these CrLIP1 mutants had reduced FARE compared to WT CrLIP1, while not severely diminishing total retinoid production.

TABLE 7
retinoid production in strain ML18743 (deletion of LIP2-3-8) compared
to wild-type strain ML18812 (“LIP+”) as control with or
without addition of the respective mutants of CrLIP1 as indicated.
“% FARE” is based on total retinoids produced with addition
of each individual lipase. “Total retinoids (% of LIP+)”
is the percentage of retinoids produced compared to total retinoids
in the control without addition of lipases, wherein the total retinoids
obtained with the control are set to 100%. For more explanation, see text.

| | | Total | |
|---|---|---|---|
| | | retinoids | |
| Strain | Lipase | (% of LIP+) | % FARE |
| ML18812 | none | 100 | 88.54 |
| ML18743 | none | 4 | 0.00 |
| ML18743 | CrLIP1 | 33.29 | 15.41 |
| ML18743 | CrLIP1_F296A | 22.06 | 0.00 |
| ML18743 | CrLIP1_F296V | 38.78 | 1.61 |
| ML18743 | CrLIP1_F296I | 13.03 | 0.00 |
| ML18743 | CrLIP1_F296S | 14.63 | 0.00 |
| ML18743 | CrLIP1_F296N | 8.34 | 0.00 |
| ML18743 | CrLIP1_F296Q | 9.82 | 0.00 |
| ML18743 | CrLIP1_296A_F344W | 5.80 | 0.00 |
| ML18743 | CrLIP1_F345L | 3.34 | 0.00 |
| ML18743 | CrLIP1_F344I | 124.67 | 91.32 |
| ML18743 | CrLIP1_F448H | 26.84 | 6.42 |
| ML18743 | CrLIP1_F448A | 53.70 | 7.66 |
| ML18743 | CrLIP1_F448W | 21.82 | 2.85 |

### Example 5: Addition of Heterologous CrLIP Chimeras in Growth-Deficient LIP2-3-8-Deletion Strains of Yarrowia lipolytica

To determine if expression of CrLIP1 would complement the corn oil utilization defect of strain ML18756, we transformed it with MB9721 (SEQ ID NO:16), which encodes a chimeric fusion protein (SEQ ID NO:24) consisting of the Yarrowia lipolytica LIP2 secretion signal with the Candida rugosa LIP1 (CrLIP1) enzyme. This strain was called ML18870. Transformants were able to grow on corn oil, and produce retinoids as indicated in Table 8. Strain ML18870 was able to improve total retinoid production in the absence of added lipase, while maintaining reduced FARE as seen in ML18812.

TABLE 8
expression of YlLIP2(pre)-CrLIP1 chimera (strain ML18870) growing
on corn oil compared to addition of purified Cr lipase 1 (CrLIP1)
and compared to wild-type strain ML18812 (“LIP+”)
as control. “% FARE” is based on total retinoids produced
with addition of the respective lipase as indicated. “Total
retinoids (% of CrLIP1)” is the percentage of retinoids
produced compared to total retinoids with addition of Cr lipase
1, wherein the total retinoids obtained with CrLIP1 are set
to 100%. For more explanation, see text.

| | Relevant | | Total retinoids | |
|---|---|---|---|---|
| Strain | lipase genotype | Lipase | (% of CrLIP1) | % FARE |
| ML18812 | LIP+ | none | 300.3 | 88.54 |
| ML18756 | lip2 lip3 lip8 | none | 8.76 | 0.00 |
| ML18756 | lip2 lip3 lip8 | CrLIP1 | 100.00 | 4.42 |
| ML18870 | lip2 lip3 lip8 + | none | 45.74 | 5.76 |
| | YlLip2(pre)-CrLIP1 | | | |

1. A retinoid-producing host cell capable of retinyl acetate formation, particularly retinyl acetate-producing host cell, such as a fungal host cell, preferably oleaginous yeast cell such as e.g. Yarrowia, comprising:
(1) one or more genetic modification(s), such as reduction or abolishment, preferably abolishment, of endogenous genes encoding enzymes involved in pre-digestion of vegetable oil into glycerol and fatty acids, preferably endogenous enzymes belonging to EC class 3.1.1.-, more preferably genes encoding endogenous enzymes with esterase or lipase activity; and
(2) addition of a heterologous enzyme with lipase activity, preferably enzymes belonging to EC class 3.1.1.-, particularly enzymes with fungal lipase activity, more preferably lipase activity from Candida species, most preferably Candida rugosa lipase activity.

2. The host cell according to claim 1, wherein the expression of endogenous genes is reduced or abolished, preferably abolished, said genes encoding enzymes with activities corresponding to enzyme activities selected from the group consisting of Yarrowia LIP2, LIP3, LIP8, LIP4, and combinations thereof.

3. The host cell according to claim 1, comprising a modification in a polypeptide obtainable from Yarrowia lipolytica with at least about 50%, such as 60, 70, 80, 90, 95, 98, or 100% identity to a polypeptide selected from the group consisting of SEQ ID NO:1, 3, 5, 7, and combinations thereof.

4. The host cell according to claim 1, wherein the endogenous enzyme corresponding to Yarrowia LIP8 is reduced or abolished, preferably abolished.

5. The host cell according to claim 1, wherein the endogenous enzymes corresponding to Yarrowia LIP2, LIP3 and LIP8 is reduced or abolished, preferably abolished.

6. The host cell according to claim 1, comprising addition of a heterologous enzyme with at least about 75% identity, such as 80, 85, 90, 95, 98 or 100% identity to SEQ ID NO:9.

7. The host cell according to claim 1, comprising addition of heterologous Candida rugosa lipase 1 (CrLIP1).

8. The host cell according to claim 1, furthermore comprising one or more amino acid substitution(s) in a sequence with at least about 75%, such as 80, 85, 90, 95, 98 or 100% identity to SEQ ID NO:9, wherein the one or more amino acid substitution(s) are located at position(s) corresponding to amino acid residue(s) selected from 296, 344, 345, 448 and combinations thereof in the polypeptide according to SEQ ID NO:9 and wherein the substitute amino acid corresponding to position 296 being alanine, valine, isoleucine, serine, asparagine or glutamine, and/or wherein the substitute amino acid corresponding to position 344 being isoleucine or tryptophan, and/or wherein the substitute amino acid corresponding to position 345 being leucine, and/or wherein the substitute amino acid corresponding to position 448 being histidine, alanine or tryptophan.

9. A process for production of retinyl acetate comprising growing the host cell according to claim 1 on triglycerides, preferably vegetable oil, wherein the formation of FARE is reduced by at least about 50 to 80% compared to a process using the respective non-modified host cell.

10. A process for the reduction of conversion of retinol into FARE comprising fermentation of a host cell according to claim 1, wherein the host cell is grown on triglycerides, preferably vegetable oil, preferably wherein FARE formation is reduced by at least about 50 to 80% compared to fermentation of the respective non-modified host cell.
